(19) United States
(12) Patent Application Publication
(10) Pub. No.: US 2025/0210195 A1
(43) Pub. Date: Jun. 26, 2025

(54) COMPUTER-IMPLEMENTED METHOD AND SYSTEM FOR DETERMINING A DEGREE OF RESPIRATORY AIRFLOW

(71) Applicants: UNIVERSITEIT ANTWERPEN, Antwerpen (BE); UNIVERSITAIR ZIEKENHUIS ANTWERPEN, Edegem (BE)

(72) Inventors: Ian MCLANE, Baltimore, MD (US); Kris IDES, Antwerp (BE); Eline LAUWERS, Wilrijk (BE); Toon STAS, Antwerp (BE); Jan STECKEL, Antwerp (BE); Kim VAN HOORENBEECK, Wilrijk (BE); Stijn VERHULST, Wilrijk (BE)

(21) Appl. No.: 18/850,731

(22) PCT Filed: Mar. 24, 2023
(86) PCT No.: PCT/EP2023/057712

§ 371 Date: Sep. 25, 2024

Related U.S. Application Data
(60) Provisional application No. 63/269,908, filed on Mar. 25, 2022.

(51) Int. Cl. G16H 50/20; A61B 7/00; G16H 10/60

(52) U.S. Cl. CPC G16H 50/20; A61B 7/003; G16H 10/60

(57) ABSTRACT

A computer-implemented method for determining a degree of respiratory airflow, includes the steps of obtaining lung sound data over time including inspiration breathing sounds and expiration breathing sounds of at least one breathing cycle. The at least one breathing cycle includes an inspiration phase and an expiration phase; pre-processing said lung sound data obtaining denoised lung sound data.

### FIELD OF THE INVENTION

The present invention generally relates to a computer-implemented method and system for determining a degree of respiratory airflow.

### BACKGROUND OF THE INVENTION

It has been known for a long time that lung sounds are valuable indicators of respiratory health and disease. They contain a wealth of clinically useful information about the underlying pathophysiology, relevant for the diagnosis and the recovery progress of various pulmonary diseases, in particular of obstructive pulmonary diseases such as cystic fibrosis, asthma, chronic obstructive pulmonary disease (COPD) and bronchopulmonary dysplasia (BPD). Standard auscultation, however, is a relatively subjective process depending on the examiner's hearing and experience. In addition, no permanent records can be made, and breathing patterns or changes over time cannot be quantified. Over the last decades, electronic stethoscopes and digital signal-processing techniques have been developed to overcome these limitations. Various methods for computational analysis have been proposed in previous research to overcome these limitations, ranging from classical signal-processing methods to data-science methods that build upon features to create classifiers using deep learning strategies. At present, the majority of the automated algorithms are directed to detecting the presence of one or more adventitious respiratory sounds or a respiratory disease. Although this information can be valuable for screening and diagnosis of patients, long-term follow-up in chronic respiratory diseases requires continuous outcomes that can be evaluated over time, in particular to evaluate effectiveness of treatment, in which case it is hardly possible to rely on imaging techniques, such as for example chest computer tomography, which is the actual gold standard to evaluate for example structural airway and lung parenchymal abnormalities in cystic fibrosis, such as mucus plugging, air trapping, bronchiectasis and bronchial wall thickening.

As a particular example, cystic fibrosis (CF) is a genetic disease caused by mutations of the cystic fibrosis transmembrane conductance regulator (CFTR) gene. Defects in the CFTR protein lead to dehydration of the airway surface liquid layer, which in turn has an impact on the mucociliary transport. This impaired mucus clearance initiates and maintains a vicious cycle of obstruction, infection and inflammation with irreversible lung damage as a result. Airway clearance techniques (ACTs) contribute to the prevention and delay of disease progression by facilitating mucus transport and expectoration. Although respiratory physiotherapy is a key component of CF care, a sound evidence base is lacking, and airway clearance strategies are largely based on clinical expertise. A range of different types of techniques can be applied, but a recent Cochrane systematic review concluded that no ACT appears to be superior. One explanation for the limited evidence is the lack of adequate outcome measures to evaluate the effectiveness of respiratory physiotherapy. Methods to assess regional changes within the airways are needed to gain more insight into the mode of action of ACTs. Digital auscultation could play an important role in the detection of these acute changes, as lung sounds contain valuable information to monitor respiratory health. By digitizing the process, lung sound characteristics can be quantified and compared over time. In addition, the procedure is non-invasive and requires only minimal set-up, which allows researchers and clinicians to apply this method at bedside or outside a hospital setting.

### SUMMARY OF THE INVENTION

It is therefore an aim of the present invention to solve or at least alleviate one or more of the above-mentioned problems. In particular, the invention aims at providing a computer-implemented method for determining a degree of respiratory airflow, which can provide a relatively objective, quantified and relatively robust basis for medical practitioners to monitor disease progression and to quantify treatment effect in a non-invasive way.

To this aim, according to a first aspect of the invention, there is provided a computer-implemented method for determining a degree of respiratory airflow having the features of claim 1. In particular, the method comprises the steps of obtaining lung sound data over time. Lung sound data are to be understood as sound data caused by a particular state of the lungs, in particular by any obstruction in the lungs. Snoring sound data are not included in lung sound data since said snoring sound data do not originate from the lungs, even if they are linked to respiration. Said lung sound data include inspiration breathing sounds and expiration breathing sounds of at least one breathing cycle. A breathing cycle is defined as including one inspiration phase and one expiration phase. Said lung sound data preferably span a plurality of breathing cycles. Said lung sound data may for example be obtained by digital lung auscultation or by any other suitable technique. Said lung sound data may preferably include lung sound data recorded at a single chest location. By applying the method separately to lung sound data obtained from a single chest location at a time, results from different chest locations can be compared with respect to a degree of respiratory airflow.

The method further comprises the step of pre-processing said lung sound data obtaining denoised lung sound data. Said pre-processing may for example include deleting background noises, such as human speech, music, environmental noise. Pre-processing can further include deleting artefacts caused by motion of a lung sound data recording device with respect to a patient and/or a user, which may be present as short-time, broad-band energy burst in the data. Pre-processing said lung sound data can be performed with any suitable and well-known sound data processing techniques. As an example, a deep learning regression network trained and validated on simulated lung sounds can be used to decrease the unwanted noise levels. As a result, denoised lung sound data can be obtained, which can improve accuracy of the further claimed method steps.

The method further comprises the step of separating the denoised lung sound data into a crackle signal including discontinuous adventitious sounds and a continuous breathing signal, said breathing signal including said inspiration breathing sounds and said expiration breathing sounds including in particular pulmonary vesicular sounds. The separation of said lung sound data in said two signals, of which the crackle signal includes discontinuous sounds, and the breathing signal includes substantially continuous breathing sounds, allows a separate analysis of said signals, each of said signals having a different origin. This separation step may be performed using any known technique in the field of sound analysis.

The method further includes the step of determining a starting time point and an end time point of the inspiration phase and of the expiration phase for every breathing cycle of the at least one breathing cycle. This step can be done before or after the step of separating the denoised lung sound data into a crackle signal and a continuous breathing signal. If this determination is done before the step of separating the denoised lung data, then the determination is performed on the denoised lung sound data. If this determination is done after the step of separating the denoised lung data, then the determination of the starting time point and end time point of the inspiration/expiration phases is performed on the continuous breathing signal. However, the obtained starting time point and end time point of the inspiration phase and of the expiration phase for every breathing cycle determined on the continuous breathing signal or on the denoised lung data are also validly used for the crackle signal.

The method further comprises the step of, for the inspiration phase of every breathing cycle of the at least one breathing cycle, determining an inspiration power of the breathing signal and of the crackle signal for at least one predetermined frequency range. An inspiration power is an average power of the breathing signal or of the crackle signal during an inspiration phase, which is defined by the starting time point and the end time point of said inspiration phase previously determined and which time points are in common for the breathing signal and the crackle signal. Analogously, the method further comprises the step of, for the expiration phase of every breathing cycle of the at least one breathing cycle, determining an expiration power of the breathing signal and of the crackle signal for at least one predetermined frequency range. Analogously, an expiration power is an average power of the breathing signal or of the crackle signal during an expiration phase. Said inspiration power and said expiration power can preferably be determined for a plurality of frequency ranges. Said inspiration power and/or expiration power can for example be determined via spectral analysis and FFT on the breathing signal and/or on the crackle signal.

Finally, the method comprises the step of determining a degree of respiratory airflow based on said inspiration power of the breathing signal, said expiration power of the breathing signal, said inspiration power of the crackle signal and/or said expiration power of the crackle signal for at least one predetermined frequency range. Respiratory airflow can include any indication of how air flows due to respiration, in particular in a lung system. Said degree of respiratory airflow can help clinicians to form a kind of quantified “sound image” of a lung system, which can assist them in evaluating for example an effect of a treatment. For example, when the method is applied before and after respiratory kinesiotherapy, the method can help in objectivizing an effect of said treatment session. The same is valid for evaluating long term effects of any treatment applied in restoration of impaired lung functions.

In the step of determining an inspiration power and/or an expiration power of the breathing signal and/or of the crackle signal, the at least one predetermined frequency range can for example include one or more of the frequency ranges from substantially 100-200 Hz, substantially 200-400 Hz, substantially 400-800 Hz and substantially 800-1600 Hz. Most respiratory adventitious sounds seem to appear within said frequency ranges. More preferably, an inspiration power and an expiration power can be determined for a plurality of frequency ranges. The frequency range of more or less 200 Hz to more or less 400 Hz has for example shown to be clinically very relevant due to its relation with bronchiectasis, mucus plugging, bronchial wall thickening and air trapping on CT. The person skilled in the art will understand that frequency ranges with different limits can be used as well.

Separating the denoised lung sound data into a crackle signal and a breathing signal can for example be performed by wavelet decomposition or a wavelet packet decomposition, for example by using a suitable filter such as a wavelet packet transform-based stationary non-stationary filter.

Said degree of respiratory airflow can for example include a degree of airway clearance, of airway permeability, of airway patency and/or of airway obstruction. Depending on the clinical case, one of these expressions may be of particular interest. Determining said degree of respiratory airflow may further include determining particular characteristics of said airflow, for example a location of obstruction.

The method can preferably further comprise the step of determining an expiration to inspiration power ratio of the breathing signal for at least one predetermined frequency range, for every breathing cycle of the at least one breathing cycle. Said expiration to inspiration power ratio of the breathing signal is the ratio of the average power of the breathing signal during the expiration phase to the average power of the breathing signal during the inspiration phase. The determining of said degree of respiratory airflow can then further be based on said expiration to inspiration power ratio of the breathing signal for the at least one predetermined frequency range. Comparison with imaging techniques of lungs has shown that this power ratio is a relatively reliable parameter for determining a degree of respiratory airflow. In particular, an increased expiration to inspiration power ratio has shown to point to increased airway obstruction, so a lower degree or respiratory airflow.

The method may further comprise the step of determining an average expiration to inspiration power ratio of the breathing signal over the at least one breathing cycle for the at least one predetermined frequency range. The average expiration to inspiration power ratio of the breathing signal is the average over a plurality of breathing cycles of the different expiration to inspiration power ratios of the breathing signal determined per breathing cycle. The determining of said degree of respiratory airflow can advantageously be further based on said average expiration to inspiration power ratio of the breathing signal over the at least one breathing cycle for the at least one predetermined frequency range. This expiration to inspiration power ratio may slightly vary over a plurality of breathing cycles. By taking an average expiration to inspiration power ratio over said breathing cycles, the ratio can be relatively robust for determining the degree of respiratory airflow.

The method can further comprise the step of determining a power ratio of the inspiration power, respectively expiration power, of the breathing signal for at least one predetermined frequency range to the total inspiration power, respectively total expiration power, of the breathing signal. The determining of said degree of respiratory airflow can then be further based on said relative inspiration power, respectively relative expiration power, of the breathing signal. The total inspiration power or the total expiration power can be defined as the inspiration, respectively expiration, power over the entire frequency range, the inspiration, respectively expiration, power in itself being an average power over the inspiration, respectively expiration, phase. In this way, relative inspiration power, or a relative expiration power can be obtained. Such a relative inspiration power, or a relative expiration power, may be considered as a normalized power and can therefore be a more robust parameter which can allow for comparisons of measurements, for example between measurements from different recording devices and/or from different patients. This relative inspiration power or relative expiration power can be calculated per breathing cycle or over an entire signal, in particular by adding up inspiration power, respectively expiration power, of a plurality of breathing cycles. Said relative inspiration, respectively expiration, power can help in determining characteristics of the breathing signal. In particular, this relative inspiration, respectively expiration, power can be a metric reflecting local ventilation of the airway.

The method may further comprise the step of determining an expiration to inspiration power ratio of the crackle signal for at least one predetermined frequency range, for every breathing cycle of the at least one breathing cycle. Said expiration to inspiration power ratio of the crackle signal is the ratio of the average power of the crackle signal during the expiration phase to the average power of the crackle signal during the inspiration phase. The determining of said degree of respiratory airflow may then be further based on said expiration to inspiration power ratio of the crackle signal for the at least one predetermined frequency range. In prior art methods, only the number of crackles was generally determined. In the present method, by further analysing the crackle signal separately, in particular by determining said expiration to inspiration power ratio of the crackle signal, relative characteristics of the expiratory crackles compared to the inspiratory crackles can be determined, which can add information on the degree of respiratory airflow. As an example, said expiration to inspiration power ratio of the crackle signal may be an indication that crackles in the inspiration cycles are louder or coarser than those in the expiratory cycles, which can point to a difference in the respiratory airflow in the expiration phase compared to the inspiration phase.

The method can further comprise the step of determining an average expiration to inspiration power ratio of the crackle signal over the at least one breathing cycle for the at least one predetermined frequency range. The average expiration to inspiration power ratio of the crackle signal is the average over a plurality of breathing cycles of the different expiration to inspiration power ratios of the crackle signal determined per breathing cycle. The determining of said degree of respiratory airflow can then further be based on said average expiration to inspiration power ratio of the crackle signal over the at least one breathing cycle for the at least one predetermined frequency range. An average power ratio over a plurality of breathing cycles can provide a relatively robust power ratio.

The method can further comprise the step of determining a power ratio of the inspiration power, respectively expiration power, of the crackle signal for at least one predetermined frequency range to the total inspiration power, respectively expiration power, of the crackle signal. Total inspiration power, respectively expiration power is understood as power over an entire frequency range. Such a relative inspiration power, or a relative expiration power, may be considered as a normalized power and can therefore be a more robust parameter which can allow for comparisons of measurements, for example between measurements from different recording devices and/or from different patients. The determining of said degree of respiratory airflow may then be further based on said relative inspiration power, respectively relative expiration power, of the crackle signal. In particular, the resulting relative band power can reflect coarseness of the crackles, i.e. energy in higher frequency ranges reflect fine crackles, while energy in lower frequencies reflect coarse crackles. Fineness or coarseness of crackles may further be related to a type and/or location of obstruction: relatively fine crackles may be caused by impeded airflow in relatively small airway passages, and coarse crackles may be caused by impeded airflow in relatively large airway passages in the lungs. Characterization of crackles can provide physicians and health providers more insight into the pathophysiological processes that generate these crackles.

The method may further comprise the step of determining a number of crackle peaks in the crackle signal per breathing cycle and/or per inspiration phase, respectively expiration phase. The determining of said degree of respiratory airflow can then be further based on said number of crackle peaks. The number of crackles can for example be associated with multiple structural abnormalities as well as with regional airway resistance as determined by functional respiratory imaging. Additionally, an average relative timing of crackles may be determined for the inspiration phase and the expiration phase.

According to a further aspect of the invention, there is provided a system for determining a degree of respiratory airflow over time having the features of claim 12. Such a system can provide one or more of the above-mentioned advantages.

According to a further aspect of the invention, there is provided a controller comprising at least one processor and at least one memory including computer program code, the at least one memory and computer program code configured to, with the at least one processor, cause the controller to perform the method as described above.

According to a further aspect of the invention, there is provided a computer program product comprising computer-executable instructions for performing the method as described above when the program is run on a computer.

According to a further aspect of the invention, there is provided a computer readable storage medium comprising computer-executable instructions for performing the method as described above the program is run on a computer.

### BRIEF DESCRIPTION OF THE DRAWINGS

FIG. 1 shows a schematic graph of a preferred embodiment of a system for determining a degree of respiratory airflow over time according to an aspect of the invention;

FIGS. 2a and 2b show a schematic graph of a preferred embodiment of the computer-implemented method for determining a degree of respiratory airflow according to an aspect of the invention;

FIG. 3A-3B show expiration-to-inspiration signal power ratio within different frequency ranges;

FIG. 4A-4D show relative band power of the discontinuous adventitious sound signal within different frequency ranges; and

FIG. 5 shows a computing system suitable for performing various steps of the method for processing a plurality of camera images of a human body target area over time according to an aspect of the invention.

### DETAILED DESCRIPTION OF EMBODIMENT(S)

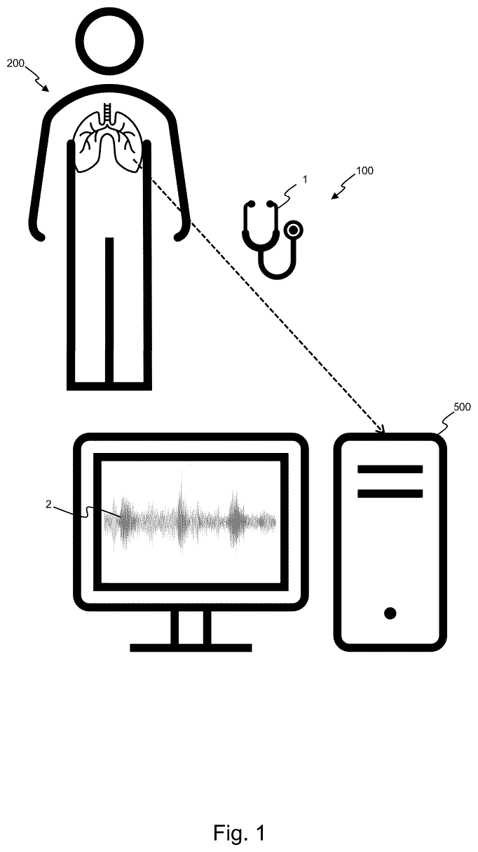

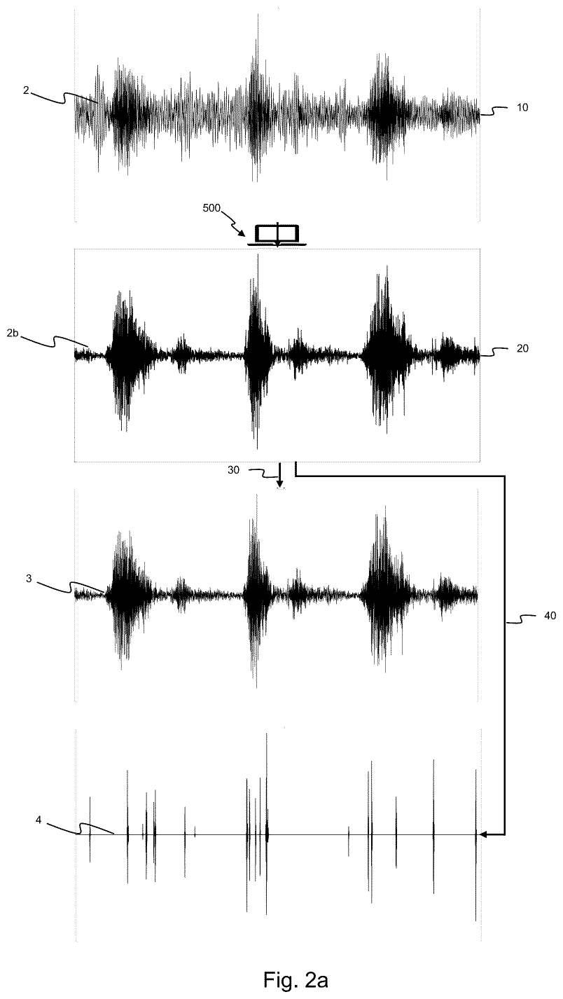

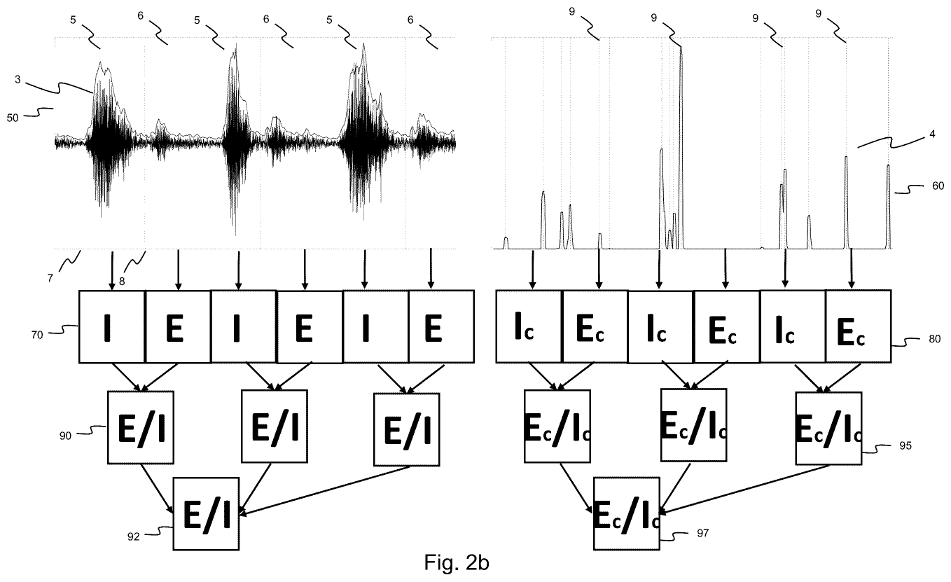

FIG. 1 shows a schematic graph of a preferred embodiment of a system for determining a degree of respiratory airflow over time according to an aspect of the invention. The system 100 comprises a respiratory sound recording device 1 configured to digitally capture lung sound data 2 over time. Such a device 1 can for example be a digital stethoscope which can be manipulated for lung auscultation by a medical caretaker or by a patient 200 himself. The system 100 further comprises a controller and a memory with computer program code configured to perform the method described above and with respect to FIG. 2. In particular, the system 100 can comprise a suitable computing system 500 described and shown with respect to FIG. 3. The computing system is configured to obtain lung sound data 2 over time, for example from said respiratory sound recording device 1.

FIGS. 2a and 2b show a schematic graph of a preferred embodiment of the computer-implemented method for determining a degree of respiratory airflow according to an aspect of the invention. The method for determining a degree of respiratory airflow comprises a first step 10 of a computer system 500 obtaining lung sound data 2 over time. The lung sound data 2 include inspiration breathing sounds and expiration breathing sounds of at least one breathing cycle, preferably of a plurality of breathing cycles. The lung sound data 2 are represented as a sound power level in arbitrary units over time, for example in seconds. One breathing cycle includes an inspiration phase and an expiration phase. In a next step 20, said lung sound data 2 are being pre-processed. Said pre-processing can for example include denoising the sound data. The sound data may be affected for example by background noises or by motion artefacts caused by motion of the respiratory sound recording device 1 during recording. These unwanted components can be deleted from the lung sound data obtaining denoised lung sound data 2b. Any suitable known method can be used for said pre-processing step 20. Then the denoised lung sound data 2b are separated 30, 40 into a crackle signal 4 including discontinuous adventitious sounds and a continuous breathing signal 3. Said breathing signal includes said inspiration breathing sounds and said expiration breathing sounds. This separation steps 30, 40 can for example be performed by implementing a wavelet or a wavelet packet filter. This separation step 30, 40 can facilitate the next step 50, the determination of a starting time point and an end time point of the inspiration phase I and of the expiration phase E for every breathing cycle. In FIG. 2b, the continuous breathing signal 3 includes three breathing cycles, each including an inspiration phase 5 followed by an expiration phase 6. For each of these inspiration phases 5 and expiration phases 6, a starting time point 7 and an end time point 8 is determined, an end time point of an expiration phase being at the same time a starting time point of the following inspiration phase. In step 70, inspiration power I of the breathing signal, which is the average power of the breathing signal per inspiration phase 5, is determined for at least one predetermined frequency range, for example for the range of more or less 200 Hz to more or less 400 Hz, or for any other predetermined frequency range. The same is done for expiration power E of the breathing signal per expiration phases 6. Optionally, in step 90, an expiration to inspiration power ratio E/I of the breathing signal may be determined per breathing cycle for at least one predetermined frequency range. In steps 60, 80 and 95, the crackle signal 4 may be processed in analogy with the breathing signal. In step 80, an inspiration power I_{c} of the crackle signal, which is the average power of the crackle signal per inspiration phase 5, is determined for at least one predetermined frequency range, for example for the range of more or less 200 Hz to more or less 400 Hz, or for any other predetermined frequency range. Thereto, inspiration and expiration phases 5, 6 are defined by the same starting and end time points, 7 and 8 determined on the corresponding breathing signal 3. The same is done for the expiration power E_{c} of the crackle signal per expiration phases 6. An additional but optional step 60 is the detection of a number of crackles 9 in the crackle signal 4 and/or the determining of a timing of an individual crackle 9. Optionally, in step 95, an expiration to inspiration power ratio E_{c}/I_{c} of the crackle signal may be determined per breathing cycle for at least one predetermined frequency range. Preferably, steps 70, 80, 90 and 95 are repeated for a plurality of frequency ranges. Finally, in optional steps 92 and 97, an average expiration to inspiration power ratio E/I of the breathing signal and an average expiration to inspiration power ratio E_{c}/I_{c} of the crackle signal may be determined for at least one predetermined frequency range. Said average is an average expiration to inspiration power ratio E/I taken over a plurality of breathing cycles. Alternatively, a mean inspiration, respectively expiration, power of the breathing signal or of the crackle signal may be determined by taking an average of the inspiration, respectively expiration, power of the breathing signal or of the crackle signal over the plurality of breathing cycles, before determining an expiration to inspiration power ratio of said mean values of the inspiration, respectively expiration, power of the breathing signal or of the crackle signal.

In a final step, which is not shown, a degree of respiratory airflow based on the inspiration power I of the breathing signal, the expiration power E of the breathing signal, the inspiration power I_{c} of the crackle signal and the expiration power E_{c} of crackle signal for at least one predetermined frequency range, per breathing cycle or averaged out over a plurality of breathing cycles, is determined. As an example, comparative studies have shown that an decrease in a power ratio E/I of the breathing signal can point to an improved respiratory airflow. In this way, a relatively objective way of evaluating effect of a treatment over time can be provided, for example by applying said method before and after respiratory physiotherapy, in particular with patients suffering from cystic fibrosis, bronchoconstriction, or any airway inflammation.

A study has been carried out to show that the above-mentioned lung sound characteristics can be a sensitive marker to measure treatment effects of airway clearance techniques in stable cystic fibrosis patients. Two treatment modalities have been compared to assess differences between types of treatment. The randomized cross-over study was conducted to assess the short-term effects of two airway clearance regimens, standard airway clearance therapy (STD) with or without the addition of intrapulmonary percussive ventilation (IPV). Treatment sessions were performed on two consecutive days in a randomized order determined by a computer program stratified by age, gender and percentage predicted of the forced expiratory volume in one second (ppFEV1). Visit A consisted of a 30-minute control period followed by 30 minutes of STD. At visit B, the STD session was preceded by 15 minutes of IPV treatment (IPV+STD). Lung sounds and secondary outcomes were assessed before and after both treatment sessions, and additional lung sound recordings were made before the control period. Participants were recruited at six primary care physiotherapy practices in Belgium between July 2018 and December 2019. Subjects were eligible for inclusion if they met the following criteria: CF diagnosis, age >5y and clinically stable at inclusion. Exclusion criteria were cognitive impairment and comorbidities that would interfere with ACTs. All treatments were performed by the patients' regular physiotherapist. Participating therapists were qualified respiratory physiotherapists and performed treatments according to the same principles. The session consisted of autogenic drainage (AD), which is a controlled breathing technique that uses the effects of shearing forces induced by airflow to increase mucus mobilization. In order to do this, inspiratory and expiratory airflow and lung volume are modulated according to the localization of the secretions. A description of the technique can be found in the IPG/CF booklet on physiotherapy techniques. To facilitate sputum expectoration, AD was combined with either positive expiratory pressure (PEP), oscillating positive expiratory pressure (OPEP) or non-invasive ventilation (NIV), depending on the patients' routine treatment. At visit B, STD was preceded by 15 minutes of IPV guided by a physiotherapist. A Travel Air® (Percussionaire Corporation, Sandpoint, Idaho, USA) was used as the percussive ventilator. A minimum frequency of 300 Hz and a working pressure between 10-20 cmH2O were maintained in accordance with recommendations made by Riffard and Toussaint for patients with obstructive lung diseases. The exact settings were selected depending on the patient's comfort and movement of the chest. Normal saline aerosol (NaCl 0.9%) was delivered to prevent dehydration of the mucosa.

Study assessments were performed by the same researcher in a consistent order before and after treatment: (1) pulse oximetry, (2) lung sound recordings, and (3) spirometry. Spirometry was performed using a portable spirometer (Spirostik, Geratherm, Germany) according to ERS standards. Peripheral oxygen saturation (SpO2) was measured by pulse oximetry. A digital stethoscope (Thinklabs One, Thinklabs Medical LLC) was used to record lung sounds over six chest locations: two posterior basal, two anterior (second intercostal space, mid-clavicular line) and two lateral (4th-5th intercostal space, mid-axillary line). Recordings were acquired using a digital stethoscope with a 32-bit resolution and a sampling rate of 44.1 kHz. Each recording was made for 25-30 seconds with the patient in a sitting position, while breathing through the mouth. The procedure was performed in accordance with the CORSA guidelines for short-term acquisition. In general, automated computer aided lung sound analysis included four main components: (1) pre-processing and denoising steps, (2) wavelet packet decomposition to separate discontinuous adventitious sounds (DAS) from the signal, (3) respiratory cycle extraction, and (4) crackle peak detection. In particular, the signal was first low-pass filtered at 4 kHz and resampled to 8 kHz to simplify processing and remove unnecessary information. Also motion artifacts presented as short-time, broad-band energy bursts were removed from the signal. A deep learning regression network trained and validated on simulated lung sounds was applied to decrease the unwanted noise levels. Discontinuous adventitious lung sounds (DAS) were separated from pulmonary vesicular sounds (PVS) using a wavelet packet transform-based stationary non-stationary filter. Next, respiratory cycle phases were identified from the PVS signal using the Hilbert transform for envelope extraction and recording-specific thresholds. The average signal power was calculated from the PVS signal during in- and expiration to obtain the expiration-to-inspiration (E/I) power ratio for different frequency bands: 100-200 Hz, 200-400 Hz, 400-800 Hz and 800-1600 Hz. The approach to identify crackles was similar to the extraction of the respiratory phases in the PVS signal, i.e. the Hilbert transform was used to calculate the signal envelope of the DAS signal and the crackle peaks were detected using a recording-specific threshold. A semi-automated approach was applied for the respiratory cycle detection to allow visual corrections if needed. The signal power during in- and expiration was determined to calculate expiration-to-inspiration (E/I) ratios for different frequency bands, i.e. 100-200 Hz, 200-400 Hz, 400-800 Hz, and 800-1600 Hz. In- and expiratory breath signal power within different frequency ranges were obtained to gain a better understanding of the behaviour of these sounds. As such, an increased E/I ratio indicates diminished inspiratory breath sounds or harsher prolonged expiratory sounds, called bronchial breathing. The average crackle count was calculated per respiratory phase. To obtain more insight into the crackle characteristics, the relative band power in the four respective frequency bands was calculated after separating the individual crackle signals from the DAS signal. The resulting relative band power reflects the coarseness of the crackles, i.e. energy in higher frequency ranges reflect fine crackles, while energy in lower frequencies reflect coarse crackles. Also, the average relative timing of the crackles was determined for both respiratory phases. Statistical analyses were performed in R for statistical computing (version 4.1.0, R Core Team 2021, Austria). Histograms and QQ plots were computed to evaluate the distribution of the data. Normally distributed data are presented as mean±standard deviation, and non-normal data as median [range]. For lung sound analysis parameters, linear mixed-effects models were computed with subject and chest location as random effects to assess treatment effects and to differentiate between types of treatment. Least square means were calculated for each factor level to allow comparison between treatments. Paired samples t-test or the Wilcoxon matched pairs test (depending on the distribution of the data) were applied for secondary outcomes. A convenience sample of 20 participants was recruited since no prior information about the expected effect size and sample variance was available for these novel lung sound analysis outcomes. Statistical significance was accepted when P<0.05.

A total of 20 (1M/19F) patients with CF were enrolled in the study and all of them completed both study visits. An equal number of subjects (n=10) was randomly allocated to treatment sequence AB or BA. Forty-five % used an OPEP device during STD as an aid to facilitate mucus mobilization, 40% PEP and 20% combined their treatment with NIV. The latter group of patients using NIV had an ppFEV1 between 21-38% at baseline. This technique was needed to prevent fatigue and dyspnea during therapy. Six recordings at different chest locations were made per subject at five time points, which resulted in a total of 600 recordings to analyse. A summary of results for all computer aided lung sound analysis parameters at each time point can be found in Table 2.

TABLE 2
Computer aided lung sound analysis results per time point.

| | | | | |
|---|---|---|---|---|
| T_{A}0 | T_{A}1 | T_{A}2 | T_{B}1 | T_{B}2 |

E/I ratio

| | | | | | |
|---|---|---|---|---|---|
| 100-200 Hz | 0.18 | 0.20 | 0.17 | 0.21 | 0.14 |
| | [0.03; 5.81] | [0.03; 5.12] | [0.02; 10.67] | [0.03; 11.82] | [0.02; 25.63] |
| 200-400 Hz | 0.09 | 0.09 | 0.08 | 0.09 | 0.07 |
| | [0.01; 3.02] | [<0.01; 2.52] | [0.01; 6.09] | [0.01; 6.15] | [<0.01; 4.70] |
| 400-800 Hz | 0.26 | 0.17 | 0.15 | 0.18 | 0.15 |
| | [0.01; 9.99] | [0.01; 14.56] | [<0.01; 95.20] | [0.01; 42.82] | [0.01; 363.14] |
| 800-1600 Hz | 1.24 | 0.75 | 0.88 | 1.02 | 0.77 |
| | [0.01; 51.97] | [0.02; 38.84] | [<0.01; 344.20] | [0.03; 118.55] | [0.04; 459.08] |

Crackle count (n)

| | | | | | |
|---|---|---|---|---|---|
| Inspiration | 1.85 | 2.23 | 1.93 | 2.00 | 2.73 |
| | [0.00; 11.00] | [0.00; 9.33] | [0.00; 10.00] | [0.00; 8.25] | [0.13; 11.67] |
| Expiration | 2.67 | 2.50 | 2.50 | 3.00 | 2.67 |
| | [0.14; 16.67] | [0.00; 16.33] | [0.00; 16.00] | [0.14; 14.00] | [0.00; 10.33] |

Relative band power DAS (%)

| | | | | | |
|---|---|---|---|---|---|
| 100-200 Hz - | 33.16 ± 5.98 | 33.82 ± 6.10 | 33.02 ± 6.36 | 31.68 ± 6.88 | 33.92 ± 5.51 |
| Inspiration | | | | | |
| 100-200 Hz - | 40.46 ± 5.49 | 40.15 ± 6.39 | 40.81 ± 5.96 | 39.84 ± 6.49 | 42.86 ± 4.89 |
| Expiration | | | | | |
| 200-400 Hz - | 44.14 ± 5.44 | 44.90 ± 6.16 | 44.47 ± 6; 41 | 45.45 ± 7.54 | 42.65 ± 4.06 |
| Inspiration | | | | | |
| 200-400 Hz - | 47.28 ± 5.60 | 49.72 ± 7.00 | 47.85 ± 6.24 | 49.14 ± 7.43 | 46.99 ± 5.68 |
| Expiration | | | | | |
| 400-800 Hz - | 8.80 | 6.85 | 7.51 | 7.68 | 7.30 |
| Inspiration | [0.22; 38.79] | [0.20; 83.91] | [0.29; 37.33] | [0.60; 31.30] | [0.29; 29.23] |
| 400-800 Hz - | 2.91 | 2.63 | 2.11 | 2.92 | 2.15 |
| Expiration | [0.28; 40.44] | [0.41; 24.02] | [0.31; 47.65] | [0.11; 23.13] | [0.18; 55.44] |
| 800-1600 Hz - | 1.91 | 1.13 | 1.24 | 1.40 | 1.42 |
| Inspiration | [0.03; 22.51] | [0.04; 17.84] | [0.03; 70.67] | [0.03; 44.87] | [0.03; 23.44] |
| 800-1600 Hz - | 0.21 | 0.17 | 0.15 | 0.21 | 0.13 |
| Expiration | [0.02; 12.34] | [0.02; 14.02] | [0.02; 12.84] | [0.01; 8.90] | [0.01; 8.53] |

Relative timing crackles (%)

| | | | | | |
|---|---|---|---|---|---|
| Inspiration | 48.22 ± 6.88 | 48.37 ± 8.33 | 47.66 ± 7.74 | 50.22 ± 9.14 | 47.03 ± 7.50 |
| Expiration | 52.82 ± 4.79 | 54.15 ± 5.38 | 54.08 ± 4.75 | 51.71 ± 4.60 | 52.30 ± 5.40 |

Data are presented as mean ± standard deviation or median [range], depending on the distribution of the data. Abbreviations: DAS, discontinuous adventitious sounds; E/I ratio, expiration-to-inspiration signal power ratio; IPV, intrapulmonary percussive ventilation; STD, standard bronchial drainage.

Values for all lung sound analysis parameters were compared at the start of each treatment regimen, i.e. STD, IPV+STD and the control period, to evaluate baseline differences (Table S1). Results showed no significant differences in any of the parameters.

TABLE S1
Baseline comparison computer aided
lung sound analysis parameters.

| | | | |
|---|---|---|---|
| | STD vs. | | IPV + |
| | IPV + | STD vs. | STD vs. |
| Model | STD | CTRL | CTRL |

E/I ratio

| | | | | |
|---|---|---|---|---|
| 100-200 Hz (log) | NS | — | — | — |
| 200-400 Hz (log) | NS | — | — | — |
| 400-800 Hz (log) | NS | — | — | — |
| 800-1600 Hz (log) | P = 0.029 | (p = 0.06) | (p = 0.05) | NS |

Crackle count (n)

| | | | | |
|---|---|---|---|---|
| Inspiration (sqrt) | NS | — | — | — |
| Expiration (sqrt) | NS | — | — | — |

Relative band power DAS (%)

| | | | | |
|---|---|---|---|---|
| 100-200 | NS | — | — | — |
| Hz - Inspiration | | | | |
| 100-200 | NS | — | — | — |
| Hz - Expiration | | | | |
| 200-400 | NS | — | — | — |
| Hz - Inspiration | | | | |
| 200-400 | NS | — | — | — |
| Hz - Expiration | | | | |
| 400-800 Hz - | NS | — | — | — |
| Inspiration (cr) | | | | |
| 400-800 Hz - | NS | — | — | — |
| Expiration (cr) | | | | |
| 800-1600 Hz - | NS | — | — | — |
| Inspiration (log) | | | | |
| 800-1600 Hz - | NS | — | — | — |
| Expiration (log) | | | | |

Relative timing crackles (%)

| | | | | |
|---|---|---|---|---|
| Inspiration | NS | — | — | — |
| Expiration | NS | — | — | — |

P-values of the mixed-effects models are presented. Abbreviations: cr, cube root; CTRL, control; DAS, discontinuous adventitious sounds; E/I ratio, expiration-to-inspiration signal power ratio; IPV, intrapulmonary percussive ventilation; NS, not significant; sqrt, square root; STD, standard bronchial drainage.

| | P-value | | | |
|---|---|---|---|---|
| | model | A - B | A - C | B - C |
| bpVesEIRdBAvg_100_200 Hz | NS | — | — | — |
| bpVesEIRdBAvg_200_400 Hz | NS | — | — | — |
| bpVesEIRdBAvg_400_800 Hz | NS | — | — | — |
| bpVesEIRdBAvg_800_1600 Hz | P = | (p = | (p = | NS |
| | 0.029 | 0.06) | 0.05) | |
| AvgNumCrackles (sqrt) | NS | — | — | — |
| AvgNumCracklesInsp | NS | — | — | — |
| AvgNumCracklesExp | NS | — | — | — |
| rbpDas_100_200 Hz | NS | — | — | — |
| rbpDasInsp_100_200 Hz | NS | — | — | — |
| rbpDasExp_100_200 Hz | NS | — | — | — |
| rbpDas_200_400 Hz | NS | — | — | — |
| rbpDasInsp_200_400 Hz | NS | — | — | — |
| rbpDasExp_200_400 Hz | (p = | NS | (p = | NS |
| | 0.054) | | 0.054) | |
| rbpDas_400_800 Hz (cr) | NS | — | — | — |
| rbpDasInsp_400_800 Hz (cr) | NS | — | — | — |
| rbpDasExp_400_800 Hz (cr) | NS | — | — | — |
| rbpDas_800_1600 Hz (log) | NS | — | — | — |
| rbpDasInsp_800_1600 Hz | NS | — | — | — |
| (log) | | | | |
| rbpDasExp_800_1600 Hz | NS | — | — | — |
| (log) | | | | |
| AvgRelCracklePosInsp | NS | — | — | — |
| AvgRelCracklePosExp | NS | — | — | — |
| (square) | | | | |

Pre-versus post-treatment measurements were compared, of which the results are presented in Table 3.

TABLE 3
Computer aided lung sound analysis results:
PRE versus POST measurements.

| | | |
|---|---|---|
| STD | IPV + STD | CTRL |

E/I ratio

| | | | |
|---|---|---|---|
| 100-200 Hz (log) | NS | P < 0.001 | NS |
| 200-400 Hz (log) | NS | P < 0.001 | NS |
| 400-800 Hz (log) | NS | P < 0.001 | NS |
| 800-1600 Hz (log) | NS | P = 0.003 | P = 0.025 |

Crackle count (n)

| | | | |
|---|---|---|---|
| Inspiration (sqrt) | NS | NS | NS |
| Expiration (sqrt) | NS | P = 0.034 | NS |

Relative band power DAS (%)

| | | | |
|---|---|---|---|
| 100-200 | NS | NS | NS |
| Hz - Inspiration | | | |
| 100-200 | NS | P = 0.009 | NS |
| Hz - Expiration | | | |
| 200-400 | NS | P = 0.042 | NS |
| Hz - Inspiration | | | |
| 200-400 | NS | P = 0.048 | P = 0.020 |
| Hz - Expiration | | | |
| 400-800 Hz - | NS | NS | NS |
| Inspiration (cr) | | | |
| 400-800 Hz - | NS | P = 0.030 | NS |
| Expiration (cr) | | | |
| 800-1600 Hz - | NS | NS | NS |
| Inspiration (log) | | | |
| 800-1600 Hz - | NS | P = 0.034 | NS |
| Expiration (log) | | | |

Relative timing crackles (%)

| | | | |
|---|---|---|---|
| Inspiration | NS | P = 0.044 | NS |
| Expiration | NS | NS | NS |

P-values of the mixed-effects models are presented. Abbreviations: cr, cube root; CTRL, control; DAS, discontinuous adventitious sounds; E/I ratio, expiration-to-inspiration signal power ratio; IPV, intrapulmonary percussive ventilation; NS, not significant; sqrt, square root; STD, standard bronchial drainage.

Only the combined treatment, IPV+STD, showed multiple significant differences. E/I ratios within all frequency bands, except for the highest frequency band 800-1600 Hz, and the number of expiratory crackles showed a significant decrease. When considering crackle characteristics before and after IPV+STD, band power at 100-200 Hz during expiration increased, while the power at 200-400 Hz decreased. In the higher frequency ranges a significant decrease was found during expiration. Also, a small significant difference in timing of the inspiratory crackles occurred after this treatment regimen. For STD without the addition of IPV, no differences were found in any of the computer aided lung sound analysis parameters. In contrast, a significant change was found in one of the parameters after the control period, namely the relative band power of the crackles at 200-400 Hz during expiration.

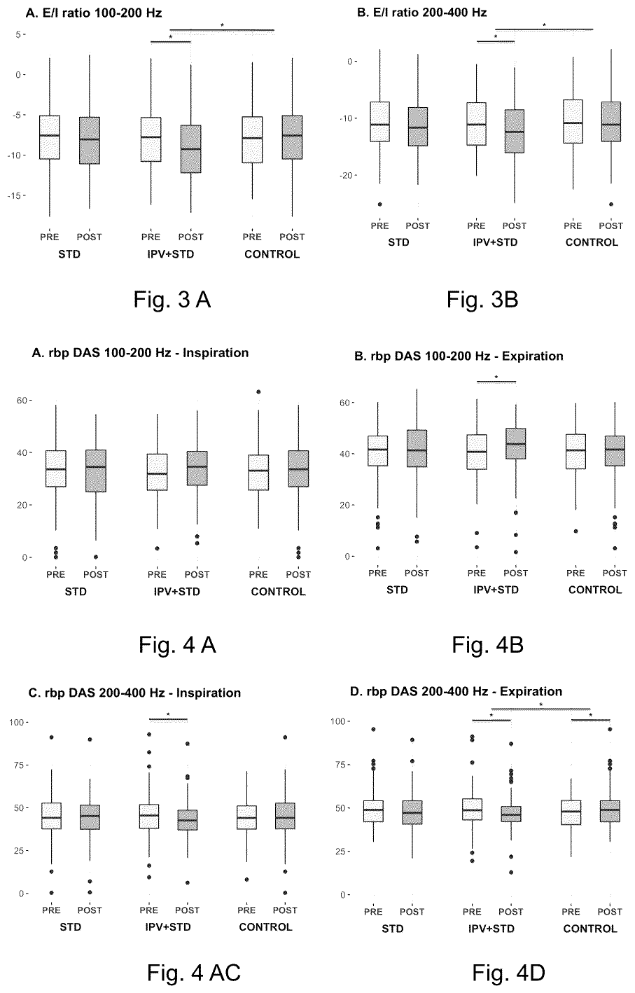

Comparing treatment modalities, the mixed-effects models showed significant differences in the changes of E/I ratios 100-200 Hz and 200-400 Hz after IPV+STD compared to the control period, as shown in FIGS. 3A and 3B visualizing expiration-to-inspiration signal power ratio within different frequency ranges. Statistically significant differences have been indicated with an asterisk *. (IPV stands for intrapulmonary percussive ventilation; STD for standard airway clearance therapy). Although the relative power of the crackles changed significantly in several frequency bands after IPV+STD, only the power between 200-400 Hz showed a significant greater change compared to the control period. This can be seen in FIGS. 4A to 4D showing relative band power of the Discontinuous Adventitious Sound Signal (DAS) within different frequency ranges. An overview of these results can be found in Table 4.

TABLE 4
Computer aided lung sound analysis results:
comparison between treatments.

| | | | |
|---|---|---|---|
| | STD vs. | | IPV + |
| | IPV + | STD vs. | STD vs. |
| Model | STD | CTRL | CTRL |

E/I ratio

| | | | | |
|---|---|---|---|---|
| 100-200 Hz (log) | P = 0.005 | NS | NS | P = 0.003 |
| 200-400 Hz (log) | P = 0.027 | NS | NS | P = 0.020 |
| 400-800 Hz (log) | NS | — | — | — |
| 800-1600 Hz (log) | P = 0.042 | (p = 0.055) | NS | NS |

Crackle count (n)

| | | | | |
|---|---|---|---|---|
| Inspiration (sqrt) | NS | — | — | — |
| Expiration (sqrt) | NS | — | — | — |

Relative band power DAS (%)

| | | | | |
|---|---|---|---|---|
| 100-200 Hz - | NS | — | — | — |
| Inspiration | | | | |
| 100-200 Hz - | NS | — | — | — |
| Expiration | | | | |
| 200-400 Hz - | NS | — | — | — |
| Inspiration | | | | |
| 200-400 Hz - | P = 0.003 | NS | P = 0.006 | P = 0.015 |
| Expiration | | | | |
| 400-800 Hz - | NS | — | — | — |
| Inspiration (cr) | | | | |
| 400-800 Hz - | NS | — | — | — |
| Expiration (cr) | | | | |
| 800-1600 Hz - | NS | — | — | — |
| Inspiration (log) | | | | |
| 800-1600 Hz - | NS | — | — | — |
| Expiration (log) | | | | |

Relative timing crackles (%)

| | | | | |
|---|---|---|---|---|
| Inspiration | NS | — | — | — |
| Expiration | NS | — | — | — |

P-values of the mixed-effects models are presented. Abbreviations: cr, cube root; CTRL, control; DAS, discontinuous adventitious sounds; E/I ratio, expiration-to-inspiration signal power ratio; IPV, intrapulmonary percussive ventilation; NS, not significant; sqrt, square root; STD, standard bronchial drainage.

Since the direction of change in crackle parameters could have been inconsistent, absolute differences were considered as well. However, this approach did not lead to any complementary findings. No significant changes were found after either STD alone or IPV+STD for FEV1, FVC, MEF25 and MEF25-75. Only a significant increase in PEF was found after IPV+STD. The changes in lung function were similar between both treatment sessions. Also, SpO2 did not change after either STD alone or IPV+STD. A summary of the results of the secondary outcomes can be found in Table 5.

TABLE 5
Results secondary outcomes

| | | | | | | | STD vs |
|---|---|---|---|---|---|---|---|
| | STD | | | IPV + STD | | | IPV + STD |
| | | | P | | | P | P |
| | PRE | POST | value | PRE | POST | value | value |
| FEV₁ | 61.20 ± 22.36 | 61.16 ± 23.37 | 0.912 | 61.15 ± 22.93 | 61.32 ± 23.02 | 0.719 | 0.645 |
| (% pred) | | | | | | | |
| FVC | 76.90 ± 17.47 | 77.05 ± 17.03 | 0.434 | 77.05 ± 18.07 | 77.89 ± 17.23 | 0.306 | 0.885 |
| (% pred) | | | | | | | |
| MEF₂₅ | 42.3 ± 40.68 | 45.53 ± 45.62 | 0.959 | 39.20 ± 37.40 | 40.89 ± 42.51 | 0.794 | 0.765 |
| (% pred) | | | | | | | |
| MEF₂₅₋₇₅ | 39.10 ± 27.59 | 40.00 ± 29.00 | 0.509 | 38.70 ± 28.05 | 39.42 ± 29.59 | 0.797 | 0.752 |
| (% pred) | | | | | | | |
| PEF | 74.40 ± 17.43 | 77.00 ± 15.95 | 0.393 | 74.75 ± 17.01 | 78.79 ± 17.11 | 0.019* | 0.235 |
| (% pred) | | | | | | | |
| SpO₂ (%) | 95.85 ± 2.50 | 96.25 ± 2.31 | 0.335 | 96.00 ± 2.59 | 96.25 ± 2.05 | 0.544 | 0.704 |

Data are presented as mean ± standard deviation,
*statistically significant (P < 0.05).
Abbreviations: % pred, percentage predicted; FEV1, forced expiratory volume in one second; FVC, forced vital capacity; IPV, intrapulmonary percussive ventilation; MEF25, maximal expiratory flow at 25% of FVC; MEF25-75, mean expiratory flow between 25% and 75% of FVC; PEF, peak expiratory flow; SpO2, peripheral oxygen saturation; STD, standard bronchial drainage.

In this study, a novel approach for computer aided lung sound analysis was used to explore which lung sound characteristics have the potential to act as sensitive outcomes for respiratory physiotherapy in CF. The analysis was based on in- and expiratory breath sounds and crackle peak detection. This approach was adopted, since crackles are considered the dominant adventitious sound in CF and no commercial tools are yet available to detect and analyse this type of sound. In summary, a decrease in E/I signal power ratio at 100-200 Hz and 200-400 Hz were found after IPV+STD compared to control. The average number of crackles did not change after a single treatment session, but differences were found in crackle characteristics. As such, the relative band power of the crackles shifted from higher to lower frequency ranges after IPV+STD, but only the decrease in power between 200-400 Hz during expiration was significant when comparing treatment effects to normal variations after a control period. Surprisingly, these significant effects were only found for the combined treatment, IPV+STD, and not for STD alone. Overall, spirometry and pulse oximetry did not show significant changes after either treatment session, except for an increase in PEF after IPV+STD.

The E/I ratios discussed in this study, are derived from the signal power of the total lung sound signal after denoising. Normal breath sounds cover a broad frequency range with intensity peaks between 100-200 Hz and an energy drop above 300 Hz. These sounds are mainly heard during inspiration and at the start of expiration [16]. Therefore, E/I ratios in a low (100-200 Hz) and mid-frequency (200-400 Hz) range are expected to be <1. An increased E/I ratio caused by diminished inspiratory sound power can be explained by poor ventilation of the respective lung region, since the intensity of breath sounds depends on respiratory flow. On the other hand, bronchial breathing can also lead to an increased E/I ratio, which has been described to be associated with bronchoconstriction, airway inflammation and/or consolidation. Since ACTs are directed at facilitating mucus mobilization and improving ventilation, it is unlikely that these techniques will have short-term effects on morphological changes related to bronchial breathing. Therefore, the significant decrease of the E/I ratio between 100-200 Hz and 200-400 Hz after IPV+STD treatment could likely be attributed to an improvement in local ventilation. Adachi et al evaluated E/I signal power ratios to measure effects of respiratory physiotherapy in children with atelectasis. E/I ratios returned to normal after resolution of the atelectasis. This suggests that these computer aided lung sound analysis parameters can also be used to measure long-term effects of ACTs, besides short-term effects related to changes in local ventilation.

Another important finding was the significant change in crackle characteristics after IPV+STD, although the average number of crackles remained stable. Several mechanisms have been attributed to the generation of crackles, including elastic stress in the airway walls related to sudden opening and closing of collapsed airways, movement of thin secretions and rupture of fluid menisci. Previous research has demonstrated that the location of the generation of crackles in the airways influences crackle characteristics. As such, crackles generated in smaller airways will typically have a shorter duration and a higher frequency and will therefore be perceived as fine crackles. On the other hand, coarse crackles with a longer duration and lower frequency will be produced in larger airways. Our results showed a shift in crackle signal power from higher to lower frequencies during expiration, which reflects a transition to more coarse crackles. This shift might be explained by mucus displacement from peripheral to more proximal airways after IPV+STD treatment. These results are in accordance with results reported by Herrero-Cortina et al. They found an increase in expiratory coarse crackles after slow-expiratory ACTs in patients with non-CF bronchiectasis, which correlated with the quantity of expectorated mucus. Marques et al studied the short-term effects of ACTs in non-CF bronchiectasis as well, but they reported an inconsistent direction of change in duration of the crackles. Although their results were not significant on a group level, they also suggested that the duration of crackles (directly related to the type of crackles) has the potential to be used as a new outcome for clinical practice.

Both E/I ratios and crackle characteristics showed significant changes after the combined treatment IPV+STD, but these changes could not be detected after STD alone. An important difference between treatment sessions was the duration. Instead of replacing the patient's routine treatment by IPV, we chose to intensify the treatment session by adding an extra 15 minutes of IPV therapy. In addition to the duration as a possible explanation, IPV could have had a larger effect in the peripheral airways. According to Riffard and Toussaint et al, physiological benefits of IPV are the following: mobilization of secretions resulting from the oscillating pressure, recruitment of obstructed areas in the lungs, and an improved gas exchange. A study by Ides et al visualized the effects of a single IPV session in patients with COPD using functional respiratory imaging. No changes were observed in classical outcome measures, but regional changes were found in airway resistance and airway volume indicating mucus displacement. Although the addition of IPV to the patient's routine treatment consisting of autogenic drainage seemed to have significant beneficial effects, our results should be interpreted with caution. To date, no evidence suggests that IPV is superior to other types of ACTs in stable CF patients. A study conducted by Van Ginderdeuren et al with a similar study design investigated the short-term effects of autogenic drainage preceded by either saline inhalation or by IPV with saline in CF. Subsequent autogenic drainage treatment led to an increase in expectorated sputum during both treatment sessions, but no differences were found in other outcomes, including heart rate, oxygen saturation and Borg score for dyspnoea. In our study, no short-term effects were found in secondary outcomes in accordance with previous research. The results suggest that computer aided lung sound analysis could be more sensitive to detect acute changes after an intensive treatment session of ACTs than conventional outcome measures. A possible explanation for this might be that multiple recordings were made to assess local changes in the lungs, while results of both spirometry and pulse oximetry only reflect the respiratory system as a single unit.

The potential added value of this new approach could be demonstrated, but caution must be applied, since the methodology inevitably shows some limitations. For instance, the STD session was not standardized across study patients, which prevents us from drawing any firm conclusions regarding the effectiveness of specific treatment modalities. Since the primary aim of this study was to assess whether computer aided lung sound analysis is sensitive to detect acute changes in the lungs, it was decided to adopt the patient's routine treatment consisting of autogenic drainage provided by the patient's regular physiotherapist to ensure optimal treatment on an individual level. Next, a heterogeneous group of CF patients was included with varying levels of disease severity and a wide age range. ACTs could have different treatment effects depending on the pulmonary manifestations related to CF lung disease. Unfortunately, the number of participants was insufficient to allow relevant sub-analyses, and no recent data from medical imaging or bacterial cultures were available to differentiate between patients. Regarding the novel computer aided lung sound analysis approach, several additional limitations can be identified. First, airflow and volume were not standardized, although these parameters have a known impact on the generation of respiratory sounds. Instead, recordings were performed during spontaneous tidal breathing to allow this methodology to be adopted in a clinical setting. Second, the number of detected crackles is probably an overestimation, since friction and movement artifacts likely introduced bias. Both features are often incorrectly recognized as crackles because of the similar short explosive nature of the sounds. This emphasizes the importance of the control period in the study design, since the quality of the recordings will have contributed to the variability in detected crackles. Only a control period was included during visit A to minimize the duration of the study visits, but no baseline differences were found between treatment sessions.

Regarding the exploratory nature of this study, a number of suggestions for further research can be made. Our results could only find beneficial effects after an intensive treatment session of the patient's standard airway clearance therapy preceded by IPV. Additional studies should address the potential added value of IPV in the routine treatment of CF patients in a more standardized study design. Other important topics for further research are the variability of computer aided lung sound analysis parameters over time and the long-term effects of different airway clearance strategies. Overall, the added value of automated lung sound analysis to detect treatment effects in a setting with limited infrastructure should be further verified. To conclude, a novel approach for computer aided lung sound analysis was adopted to evaluate short-term treatment effects of ACTs in CF focusing on in- and expiratory sound power and crackles. Results of this study suggest that lung sound characteristics are more sensitive to detect acute changes after treatment than conventional outcome measures. An intensive treatment session, including IPV and autogenic drainage, led to a decrease in E/I ratios and a shift in crackle characteristics from finer to coarser expiratory crackles. More specifically, the decrease in E/I ratios suggests improved local ventilation after treatment, while the increase in coarseness of the crackles indicates mucus displacement from smaller to more proximal airways. The potential of computer aided lung sound analysis differentiate between types of respiratory physiotherapy and to increase the effectiveness of ACTs should be verified in further research.

In a further study, the relationship between multiple computer-aided lung sound analysis parameters and imaging biomarkers was investigated in a heterogeneous group of CF patients. Results of the statistical analysis showed significant associations between E/I power ratio between 200-400 Hz and structural abnormalities shown on CT. The average number of crackles was also associated with multiple structural abnormalities on CT and airway resistance determined by functional respiratory imaging (FRI). The intensity of normal breath sounds peaks between 100-200 Hz with an energy drop above 300 Hz. Generally, breath sounds are audible at the chest during inspiration, while the expiratory sound has a much lower intensity. In our study, in- and expiratory signal power could not be considered individually, as the digital stethoscope applies a build-in algorithm to amplify the sound of the recording depending on the overall intensity of the signal. Therefore, only the power ratios were retained to allow comparison between recordings. An increased E/I ratio can either be related to a decreased inspiratory sound or an increased expiratory sound. As sound intensity is directly related to respiratory flow, diminished inspiratory breath sounds could indicate poor ventilation of the respective lung region. On the other hand, a relative increase in expiratory sound, often named ‘bronchial breathing’, has been reported previously to be related to morphological changes of the airways and lung parenchyma resulting from bronchoconstriction, airway inflammation and/or consolidation. Therefore, it was expected that increased E/I ratios within the frequency range of normal breath sounds could be related to pulmonary disease. Although the intensity of normal breath sounds is the highest between 100-200 Hz, these sounds are mixed with cardiovascular and muscle sounds, which makes it more difficult to distinguish between sounds. This could explain why the E/I ratio in this low frequency range was not related to any imaging biomarker, nor pulmonary function. Our results suggest that E/I 200-400 Hz is the most promising parameter of the frequency band analysis to indicate the severity level of CF lung disease, as most associations were found within this range. This ratio has already been pointed out in previous research to be an indicator of airway narrowing and inflammation in asthma. As the majority of the CF-CT sub-scores were associated with E/I 200-400 Hz, the power ratio cannot be used to discriminate between different pulmonary manifestations. However, this could be due to the interdependency of abnormalities, e.g. bronchiectasis will most often be accompanied by increased mucus plugging. Only the extent of parenchymal abnormalities was not associated with E/I 200-400 Hz, although a significant result would have been expected due to changes in sound transmission following consolidation or atelectasis. In addition to the frequency band analysis, the average number of crackles also showed several significant associations with structural as well as functional abnormalities. CF lung disease is typically associated with coarse crackles that can be heard during early to mid-inspiration and to a lesser extent throughout expiration. The origin of crackles has been attributed to elastic stress in the airway walls related to sudden opening or closing of collapsed airways, movement of thin secretions and rupture of fluid menisci. Keeping these physiological principles in mind, the number of inspiratory crackles could be related to lung structure and function. The average number of crackles during both in- and expiration were significantly associated with all three FRI parameters: airway volume, airway resistance and air trapping. In contrast to all other imaging biomarkers, a negative association was found with total airway volume per lung lobe. Although bronchiectasis increases airway volume, the presence of mucus, inflammation or bronchoconstriction will result in a decreased volume, since the latter FRI parameter only considers intraluminal air. This reasoning is strengthened by the positive relation between the number of crackles and regional airway resistance.

Although these results are promising and can overall be explained from a physiological point of view, they should be interpreted with caution. An important limitation of research in the field of computerized respiratory sound analysis in general is that no gold standard is available to evaluate new automated approaches. Most algorithms at present are validated against manual annotations, but this subjective method is inevitably associated with considerable inter- and intra-observer variability. In addition, often only a relatively small dataset is feasible to annotate, preventing the algorithms to be generalized to a more heterogeneous group of subjects. For this reason, the approach applied in this study was recently validated by McLane et al using a large dataset of simulated lung sounds, such that the exact timing of respiratory phases and crackle peaks were known. The performance of the cycle extraction and crackle peak detection was 96% and 95% (expressed as F-scores), respectively. Notwithstanding the advantages of a simulated dataset, real patient data are more complex, which has an impact on the accuracy of the algorithms. Therefore, minor adjustments to respiratory cycle annotations were made as all lung sound parameters depend on the timing of the respiratory phases. Also, a link was found between the quality of the recordings and the number of crackles detected, despite the implementation of a denoising algorithm. Since crackles, friction and motion artifacts are all discontinuous explosive sounds, the latter two features are often incorrectly recognized as crackles. However, it would not have been feasible to manually review the crackle detection due to the resemblance of crackles and friction, the overlap of subsequent crackles and the dominance of louder breath sounds or ambient noise. Fortunately, the number of crackles was related to various pulmonary manifestations, which suggests that the clinical value of the detected crackles was sufficient to overcome the introduced errors.

Overall, this study offers a first clinical validation of the parameters derived from automated computer aided lung sound analysis. The continuous output values provide objective and regional information that might contribute to a better understanding of disease severity and progression. As mentioned in the introduction, CT imaging is the gold standard to evaluate the respiratory system, but this measure is only available in a hospital setting and is not suitable for frequent assessment due to the associated radiation dose. Digital auscultation, on the other hand, can be adopted in various settings, since the process is non-invasive, requires only a minimal setup and can be performed without patient cooperation. In this regard, automated computer aided lung sound analysis could be a valuable tool for CF, as this population is in need for early and intensive follow-up to minimize the negative consequences related to the vicious cycle of inflammation and infection. Furthermore, automated computer aided lung sound analysis could provide additional information to conventional pulmonary function tests (e.g. spirometry) by assessing the lungs on a regional level.

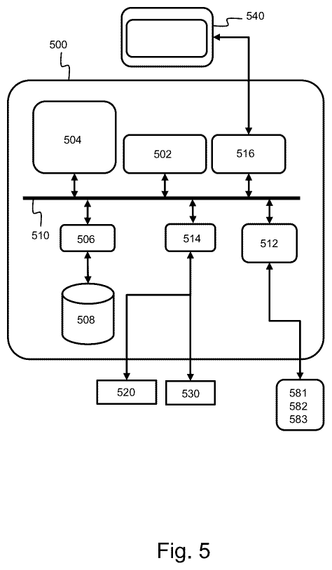

FIG. 5 shows a suitable computing system 500 comprising circuitry enabling the performance of steps of embodiments of the method for processing a plurality of camera images of a human body target area over time according to an aspect of the invention. Computing system 500 may in general be formed as a suitable general-purpose computer and comprise a bus 510, a processor 502, a local memory 504, one or more optional input interfaces 514, one or more optional output interfaces 516, a communication interface 512, a storage element interface 506, and one or more storage elements 508. Bus 510 may comprise one or more conductors that permit communication among the components of the computing system 500. Processor 502 may include any type of conventional processor or microprocessor that interprets and executes programming instructions. Local memory 504 may include a random-access memory (RAM) or another type of dynamic storage device that stores information and instructions for execution by processor 502 and/or a read only memory (ROM) or another type of static storage device that stores static information and instructions for use by processor 502. Input interface 514 may comprise one or more conventional mechanisms that permit an operator or user to input information to the computing device 500, such as a keyboard 520, a mouse 530, a pen, voice recognition and/or biometric mechanisms, a camera, etc. Output interface 516 may comprise one or more conventional mechanisms that output information to the operator or user, such as a display 540, etc. Communication interface 512 may comprise any transceiver-like mechanism such as for example one or more Ethernet interfaces that enables computing system 500 to communicate with other devices and/or systems, for example with other computing devices 581, 582, 583. The communication interface 512 of computing system 500 may be connected to such another computing system by means of a local area network (LAN) or a wide area network (WAN) such as for example the internet. Storage element interface 506 may comprise a storage interface such as for example a Serial Advanced Technology Attachment (SATA) interface or a Small Computer System Interface (SCSI) for connecting bus 510 to one or more storage elements 508, such as one or more local disks, for example SATA disk drives, and control the reading and writing of data to and/or from these storage elements 508. Although the storage element(s) 508 above is/are described as a local disk, in general any other suitable computer-readable media such as a removable magnetic disk, optical storage media such as a CD or DVD, -ROM disk, solid state drives, flash memory cards, . . . could be used.

As used in this application, the term “circuitry” may refer to one or more or all of the following:
(a) hardware-only circuit implementations such as implementations in only analogue and/or digital circuitry and
(b) combinations of hardware circuits and software, such as (as applicable):
   (i) a combination of analogue and/or digital hardware circuit(s) with software/firmware and
   (ii) any portions of hardware processor(s) with software (including digital signal processor(s)), software, and memory(ies) that work together to cause an apparatus, such as a mobile phone or server, to perform various functions) and
(c) hardware circuit(s) and/or processor(s), such as microprocessor(s) or a portion of a microprocessor(s), that requires software (e.g. firmware) for operation, but the software may not be present when it is not needed for operation.

This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in a server, a cellular network device, or other computing or network device.

Although the present invention has been illustrated by reference to specific embodiments, it will be apparent to those skilled in the art that the invention is not limited to the details of the foregoing illustrative embodiments, and that the present invention may be embodied with various changes and modifications without departing from the scope thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. In other words, it is contemplated to cover any and all modifications, variations or equivalents that fall within the scope of the basic underlying principles and whose essential attributes are claimed in this patent application. It will furthermore be understood by the reader of this patent application that the words “comprising” or “comprise” do not exclude other elements or steps, that the words “a” or “an” do not exclude a plurality, and that a single element, such as a computer system, a processor, or another integrated unit may fulfil the functions of several means recited in the claims. Any reference signs in the claims shall not be construed as limiting the respective claims concerned. The terms “first”, “second”, third”, “a”, “b”, “c”, and the like, when used in the description or in the claims are introduced to distinguish between similar elements or steps and are not necessarily describing a sequential or chronological order. Similarly, the terms “top”, “bottom”, “over”, “under”, and the like are introduced for descriptive purposes and not necessarily to denote relative positions. It is to be understood that the terms so used are interchangeable under appropriate circumstances and embodiments of the invention are capable of operating according to the present invention in other sequences, or in orientations different from the one(s) described or illustrated above.

1.-15. (canceled)

16. A computer-implemented method for determining a degree of respiratory airflow, the method comprising the steps of:
obtaining lung sound data over time including inspiration breathing sounds and expiration breathing sounds of at least one breathing cycle, the at least one breathing cycle including an inspiration phase and an expiration phase;
pre-processing said lung sound data obtaining denoised lung sound data;
separating the denoised lung sound data into a crackle signal including discontinuous adventitious sounds and a continuous breathing signal, said breathing signal including said inspiration breathing sounds and said expiration breathing sounds;
for every breathing cycle of the at least one breathing cycle, determining a starting time point and an end time point of the inspiration phase and of the expiration phase;
for the inspiration phase of every breathing cycle of the at least one breathing cycle, determining an inspiration power of the breathing signal and of the crackle signal for at least one predetermined frequency range;
for the expiration phase of every breathing cycle of the at least one breathing cycle, determining an expiration power of the breathing signal and of the crackle signal for at least one predetermined frequency range;
determining a degree of respiratory airflow based on said inspiration power of the breathing signal, said expiration power of the breathing signal, said inspiration power of the crackle signal and/or said expiration power of the crackle signal for at least one predetermined frequency range.

17. The method according to claim 16, further comprising the step of:
for every breathing cycle of the at least one breathing cycle, determining an expiration to inspiration power ratio of the breathing signal for at least one predetermined frequency range;
wherein the determining of said degree of respiratory airflow is further based on said expiration to inspiration power ratio of the breathing signal for the at least one predetermined frequency range.

18. The method according to claim 16, further comprising the step of:
determining an average expiration to inspiration power ratio of the breathing signal over the at least one breathing cycle for the at least one predetermined frequency range;
wherein the determining of said degree of respiratory airflow is further based on said average expiration to inspiration power ratio of the breathing signal over the at least one breathing cycle for the at least one predetermined frequency range.

19. The method according to claim 16, further comprising the step of:
determining a power ratio of the inspiration power, respectively expiration power, of the breathing signal for at least one predetermined frequency range to the total inspiration power, respectively total expiration power, of the breathing signal;
wherein the determining of said degree of respiratory airflow is further based on said relative inspiration power, respectively relative expiration power, of the breathing signal.

20. The method according to claim 16, further comprising the step of:
for every breathing cycle of the at least one breathing cycle, determining an expiration to inspiration power ratio of the crackle signal for at least one predetermined frequency range;
wherein the determining of said degree of respiratory airflow is further based on said expiration to inspiration power ratio of the crackle signal for the at least one predetermined frequency range.

21. The method according to claim 16, further comprising the step of:
determining an average expiration to inspiration power ratio of the crackle signal over the at least one breathing cycle for the at least one predetermined frequency range;
wherein the determining of said degree of respiratory airflow is further based on said average expiration to inspiration power ratio of the crackle signal over the at least one breathing cycle for the at least one predetermined frequency range.

22. The method according to claim 16, further comprising the step of:
determining a power ratio of the inspiration power, respectively expiration power, of the crackle signal for at least one predetermined frequency range to the total inspiration power, respectively expiration power, of the crackle signal;
wherein the determining of said degree of respiratory airflow is further based on said relative inspiration power, respectively relative expiration power, of the crackle signal.

23. The method according to claim 16, the method further comprising the step of determining a number of crackle peaks in the crackle signal per breathing cycle and/or per inspiration phase, respectively expiration phase,
wherein the determining of said degree of respiratory airflow is further based on said number of crackle peaks.

24. The method according to claim 16, wherein the at least one predetermined frequency range is one or more of the frequency ranges from substantially 100-200 Hz, substantially 200-400 Hz, substantially 400-800 Hz and substantially 800-1600 Hz.

25. The method according to claim 16, wherein separating the denoised lung sound data into a crackle signal and a breathing signal is performed by wavelet decomposition.

26. The method according to claim 16, wherein the determining of said degree of respiratory airflow includes determining airway clearance, determining airway permeability, and/or determining airway obstruction.

27. A system for determining a degree of respiratory airflow over time comprising:
a respiratory sound recording device configured to digitally capture lung sound data over time,
a controller and a memory with computer program code configured to perform the method according to claim 16.

28. A controller comprising at least one processor and at least one memory including computer program code, the at least one memory and computer program code configured to, with the at least one processor, cause the controller to perform the method according to claim 16.

29. A computer program product comprising computer-executable instructions for performing the method according to claim 16, when the program is run on a computer.

30. A computer readable storage medium comprising computer-executable instructions for performing the method according to claim 16, when the program is run on a computer.
